(19) United States
(12) Patent Application Publication
(10) Pub. No.: US 2009/0277458 A1
(43) Pub. Date: Nov. 12, 2009

(54) HEAD CAP CONDOM

(76) Inventor: Daniel J. McCarthy, Troy, MI (US)

Correspondence Address: GUBERNICK ASSOCIATES;FRANKLIN L GUBERNICK, 2540 N RISING STAR TR, TUCSON, AZ, 85745, US

(21) Appl. No.: 12/118,727

(22) Filed: May 11, 2008

(51) Int. Cl. A61F 6/04

(52) U.S. Cl. 128/844

(57) ABSTRACT

A condom configurable by a user to cover either an entire penis, just the glans penis, or the glans penis and a part of the penis' shaft. When used during fellatio, the condom preferably covers just the glans penis and thereby enables a maximum of tactile sensation. To achieve a reduced length state, the condom includes a circumferentially-extending weakened area that facilitates the separation and removal of an elongated body part of the condom from a hemispherically-shaped cap part of the condom. The cap part includes a multi-layered band of adhesive that enables its securement to the penis. An expandable backing strip may be employed to cover the band of adhesive prior to the condom's use.

### FIELD OF THE INVENTION

The invention is in the field of devices primarily used during sexual relations between humans. More particularly, the invention is a condom configurable by a user to cover either an entire penis, just the glans penis (head portion of the penis), or the glans penis and a portion of the penis' shaft.

### BACKGROUND OF THE INVENTION

A typical condom is in the form of elongated tube made of a flexible, fluid-impermeable material. At one end, the condom will have an opening through which a user can insert his penis. The other end of the condom is closed, usually dome-shaped, and may include a center-located, elongated, cylindrical reservoir sized for receiving a user's ejaculate.

Prior art condoms are normally secured to a penis through the inherent resiliency of the condom material and/or through the use of an elastic element incorporated into the condom's open end. Said elastic element may be in the form of an elastic band, or when the condom is made of an elastic material, a rolled portion of the condom. Obviously, the strength of the condom's securement will depend on the resiliency of the condom material and/or elastic element, as well as the degree to which said material and/or element is stretched when the condom is in place on a user's penis.

One problem with conventional prior art condoms is that when the condom is in place on a user's penis, the condom covers a large portion of the penis, thereby muting a user's tactile sensations during sexual relations. In an attempt to overcome this problem, many manufacturers have resorted to making their condoms as thin as possible. However, this solution is extremely limited since reducing the condom's thickness decreases its strength and increases the probability that the condom will tear.

In recent years, the prior art has taken another tack in an attempt to increase the tactile experience for a user. A few recent patents have taught the use of a reduced-length mini-condom designed to cover only the glans penis.

Blake provides one example of a mini-condom in U.S. Pat. No. 6,298,853. Phelps provides another example of a mini-condom in U.S. Pat. No. 4,821,742. In both patents, an adhesive is used to secure the condom to the glans penis. The adhesive is applied to the inner surface of the condom and is covered by a removable backing strip that is removed prior to donning the condom.

A mini-condom's greatest advantage is that it leaves most of the penis exposed. However, its greatest disadvantage is that since only a small portion of the penis is covered, there is little purchase for the mini-condom and therefore securement is a major issue. Prior art mini-condoms also have issues associated with their donning, and with their removal.

When donning a prior art mini-condom, the backing strip is normally removed prior to placement of the condom on the penis. As the condom is placed on the penis, the exposed adhesive may inadvertently become smeared on the penis. This can result in an inadequate amount of adhesive being available to secure the mini-condom on the penis and also may create a gap through which ejaculate could later flow. In addition, extra caution is required to prevent the adhesive from contacting, and possibly entering, the penis' urethral opening (urinary meatus).

When removing the condom, one breaks the engagement between the adhesive and the skin of the penis. It should be noted that the adhesive must be relatively strong since, during sexual relations, it must remain attached to the condom and also securely retain the condom on the penis. As a result, it may be very difficult to break the engagement between the adhesive and the penis without damaging the sensitive skin of the penis.

### SUMMARY OF THE INVENTION

The primary embodiment of the invention is an improved condom that can be adapted for multiple types of use and includes a cap portion and a separable body portion. The cap portion features a band of adhesive that can be engaged to a user's penis proximate the glans penis. The body portion has a length whereby it can be rolled onto, and cover, the shaft of a user's penis. The border between the condom's body portion and cap portion preferably features a circumferentially-extending weakened area whereby once said condom is at least partially in place on a user's penis, said body portion can be separated from said cap portion and removed, without removing said cap portion from the penis.

The band of adhesive is preferably a composite of two-layers of different adhesives, wherein an outer layer employs an adhesive that is specifically designed to enable removal of the backing strip and then releasable securement to the penis. The inner layer preferably employs a stronger adhesive and functions to ensure that both layers of adhesive stay secured to the condom at all times.

A backing strip made of an expandable material is used to cover the band of adhesive until the condom is partially donned. The backing strip is left in place until the condom is unrolled to the point where the glans penis is located within the cap portion of the condom. The ability to maintain the band of adhesive in a covered condition while the condom is being unrolled over the glans penis is enabled by the expandable nature of the backing strip. The strip expands with the condom material when the condom is stretched during the donning process. Once the glans penis is positioned within the condom's cap portion, the backing strip is removed and the condom is further unrolled onto the penis as the band of adhesive is pressed into contact with the penis.

Once the condom's band of adhesive is engaged to the penis, a user has two options in how to configure the condom. When a condom in accordance with the invention is to be used for intercourse, the body portion of the condom is then unrolled to cover the shaft of the penis. At this point, the band of adhesive and the elasticity of the condom's body and cap portions function to secure the condom to the penis. These multiple forms of securement not only help to ensure that the condom stays in place on the penis, but they provide increased peace of mind to the user that the user's semen will not escape from the condom during intercourse.

When a condom in accordance with the invention is to be used during fellatio, the forces on the condom tending to cause its disengagement from the penis are usually less than those experienced during intercourse. As a result, the previously described multiple forms of securement associated with the use of the full-length form of the condom is not needed. Once the condom has been donned to the point where the band of adhesive is engaged to the penis, a user can then hold the condom's cap portion in place and separate the condom's body portion from its cap portion. The weakened area where the condom's cap and body portions meet facilitates said separation. The body portion is then removed from the penis, leaving the condom's cap portion in place on the glans penis. After fellatio has been completed, the user disengages the condom's band of adhesive from the penis by preferably applying outward pressure on the end of the condom's cap portion proximate the band of adhesive.

There are a number of methods that can be employed for creating the weakened area between the condom's cap and body portions. In the preferred embodiment, the weakening of the membrane that forms the condom is achieved through physical means, such as scoring or perforations. Alternatively, the weakened area can be chemically induced via an agent, such as a weak solvent, that is applied to the portion of the condom where one wishes to weaken the condom material. If the condom is made of a heat-degradable material, localized heating of the condom material can also be used to weaken said area. Other known material weakening processes may alternatively be employed.

In a secondary embodiment of the invention, the condom's body portion includes at least one removable window portion. By removing a window portion during the donning process, a user exposes a greater area of the penis' shaft to thereby maximize potential tactile sensation.

In a third embodiment of the invention, the cap portion of the condom includes a band of adhesive and also a band of non-stick material. The non-stick material is positioned whereby when the condom is in a rolled-up condition, the condom's band of adhesive releasably contacts the non-stick material, thereby negating the need to cover the band of adhesive with a removable backing strip.

A first object of the invention is to provide a condom that is adapted to enable its use as either a full-length condom, or as a mini-condom obtained through removal of a body portion of the condom as facilitated by a weakened area of the condom.

A second object of the invention is to provide a condom that includes a medially-located band of adhesive configured and composed to enable securement of the condom's cap portion to a penis and then later allow said condom to be removed without damaging the skin of the penis.

A third object of the invention is to provide a condom that features a band of adhesive covered by a backing strip made of the same material as the condom and that can be expanded to allow its use during the donning process until the band of adhesive is properly positioned.

A fourth object of the invention is to provide a condom in which a body portion of the condom can help to press the condom's band of adhesive onto a user's penis.

A fifth object of the invention is to provide an alternate embodiment of a condom that has at least one removable portion to thereby enable a user to expose a maximum of the penis' shaft.

A sixth object of the invention is to provide an alternate embodiment of a condom in which a band of non-stick material can contact the condom's band of adhesive.

### BRIEF DESCRIPTION OF THE DRAWINGS

FIG. 1 is an end view of a condom in accordance with the invention, wherein said condom is shown in a substantially rolled state.

FIG. 2 is a cross-sectional view of the condom shown in FIG. 1, taken at the plane labeled 2-2 in FIG. 1.

FIG. 3 is a cross-sectional view of the same condom as shown in FIG. 1, but showing said condom in a first partially unrolled state, and further showing a penis in phantom.

FIG. 4 is a cross-sectional view of the same condom as shown in FIG. 1, but showing said condom in a second partially unrolled state with the backing strip removed, and further showing a penis in phantom.

FIG. 5 is a cross-sectional view of the same condom as shown in FIG. 1, but showing said condom in a third partially unrolled state with the backing strip removed, and further showing a penis in phantom.

FIG. 6 is a cross-sectional view of the same condom as shown in FIG. 1, but showing said condom in a substantially fully unrolled state with the backing strip removed, and further showing a penis in phantom.

FIG. 7 shows the same condom as shown in FIGS. 5 and 6 positioned on a penis but after the condom's body portion has been removed.

FIG. 8 is a cross-sectional view of a first alternate embodiment of a weakened portion of a condom in accordance with the invention.

FIG. 9 is a cross-sectional view of a second alternate embodiment of a weakened portion of a condom in accordance with the invention.

FIG. 10 provides a side view of another embodiment of a condom in accordance with the invention, and wherein said condom is shown positioned on a penis.

FIG. 11 shows the condom of FIG. 10 after one of its window portions has been removed.

FIG. 12 provides a cross-sectional view of an additional embodiment of a condom in accordance with the invention and wherein the condom is shown in a partially rolled-up state.

FIG. 13 provides a side view of the condom shown in FIG. 12 wherein said condom is in a further unrolled state on a penis.

FIG. 14 provides a cross-sectional view of the condom shown in FIG. 13.

### DETAILED DESCRIPTION OF THE DRAWINGS

Looking now to the drawings in greater detail, wherein like reference numerals refer to like parts throughout the several figures, there is indicated by the numeral 1 a condom in accordance with the invention.

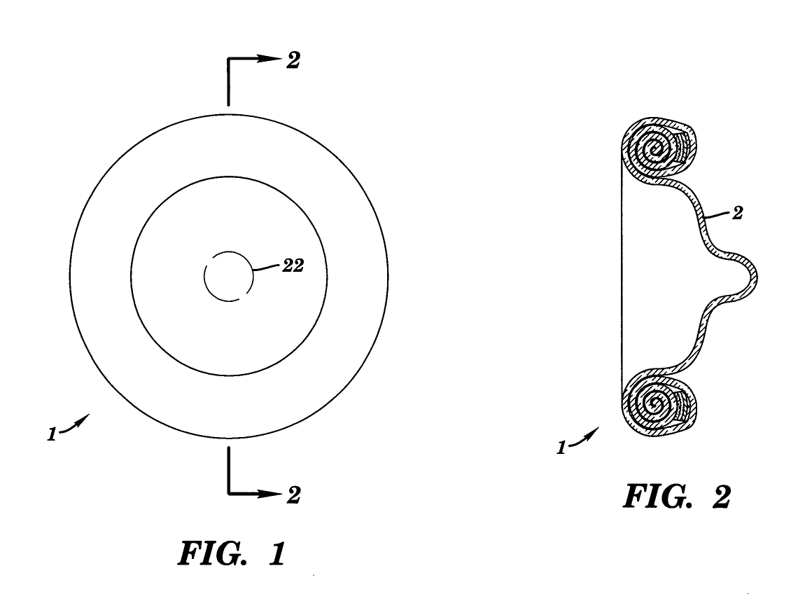

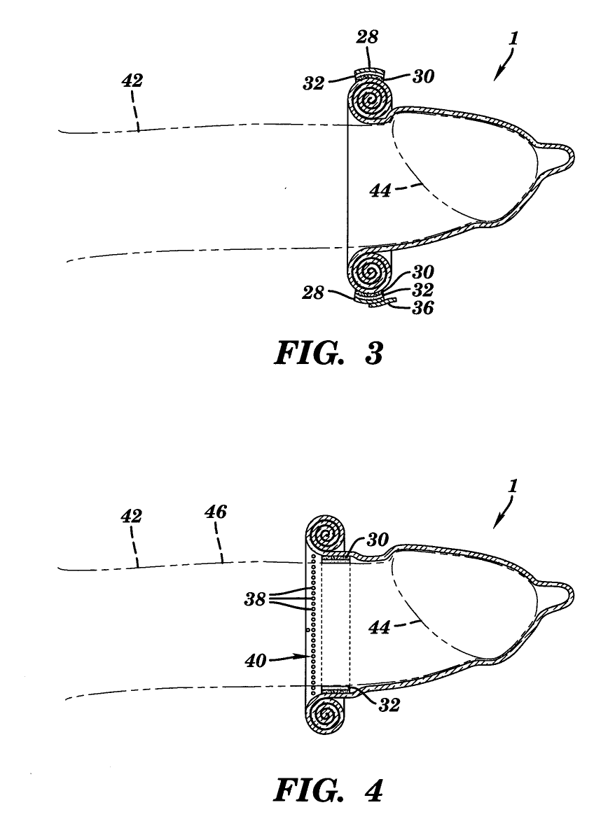

FIGS. 1-6 show the condom in consecutive states of being unrolled, with FIGS. 1 and 2 showing the condom after it has been removed from its storage packaging (not shown) whereby said condom is in a substantially fully rolled state. FIGS. 3-5 show the condom in various partially unrolled states, while FIG. 6 shows the condom in a substantially fully unrolled state.

The condom 1 is fashioned from a thin, flexible, fluid impermeable membrane 2 that is of substantially uniform thickness. The membrane material is preferably one that also has some degree of resilience, such as latex, animal intestine, or a number of plastics, including polyester. The membrane can be made of any material commonly used for the manufacture of condoms or that may be suitable for such usage.

While preferably being a unitary structure, the condom 1 may be considered to have an elongated body portion 4 (note FIG. 6) and a cap portion 6. The membrane 2 has a weakened area 8 where portions 4 and 6 meet. It should be noted that if the body and cap portions are separate pieces, only the cap portion is required to be fluid impermeable.

The body portion 4 has an open end 10 and is preferably in the form of a tube having a substantially uniform diameter. End 10 defines a large circular opening through which a user can insert a penis into the condom 1. While not shown, said end can additionally include an added elastic band to increase the strength of its removable securement to a penis.

Extending parallel to a longitudinal axis of the body portion is an optional line 12 of perforations 14. These perforations may extend partially, or completely, through the membrane 2.

The length of the body portion 4 preferably approximates the length of a shaft portion of a typical human penis. As a result, the condom's body portion will form a major portion of the condom's overall length. Alternatively, the body portion can be shorter whereby it only covers a small portion of the penis' shaft. When a shorter body portion is employed, it cannot aid in securing the condom to the penis to the same degree as can a longer body portion.

The condom's cap portion 6 is in the form of a tube or cup that is closed at one end and has a length equal to, or just slightly longer than, a typical human glans penis. In this manner, said cap portion is sized to receive the glans penis. As such, the length of portion 6 is preferably much less than the length of the condom's body portion 4, and thereby is responsible for only a minor portion of the condom's overall length. While not preferred, since a goal of the cap portion is to cover the smallest portion of a penis possible, an alternate embodiment of a condom in accordance with the invention could be made in which the cap portion is not shorter than the body portion.

Cap portion 6 is composed of first and second end portions, 16 and 18 respectively. Portion 16 is predominantly hemispherical in shape and forms the closed end 20 of the condom. Portion 18 is joined to the condom's body portion 4.

Located at the center of portion 16 is an optional, outwardly-extending cylindrical reservoir tip portion 22. The reservoir tip portion is preferably sized whereby it can receive a quantity of human semen of a volume typical for what would be expelled by a human during sexual relations.

Portion 18 of the condom's cap portion has a diameter that is substantially the same as that of the body portion and may be an extension of said body portion. Located on what will be an inwardly-facing surface 24 of portion 18, proximate said weakened area 8, is a narrow band 26 of adhesive that extends in a complete circle about said surface 24. Covering said band of adhesive is a removable backing strip 28 (note FIG. 3).

As can be seen in FIG. 3, band 26 of adhesive is preferably comprised of a first layer 30 of a first adhesive, and a second layer 32 of a second adhesive. Layer 30 contacts, and is adhered to, surface 24, while layer 32 is designed to contact the backing strip 28 and a user's skin.

Layer 30 of adhesive securely contacts surface 24 of the condom in a manner whereby it will not pull away from said surface when the backing strip is being removed and also when condom 1 is being employed during sexual relations. One such suitable adhesive is Hi-Tack Medical Transfer Adhesive #1504, made by the 3M Company, and which has a two-hundred ounce-per-inch pull strength. It should be noted that surface 24, in the area where the adhesive will be applied, might be roughened, dimpled or otherwise treated to increase adhesion between it and the adhesive.

Layer 32 of adhesive must be capable of adhering to the backing strip, allowing said backing strip to be removed, directly contacting the user's penis, and then securing the condom to the user's penis. When it is time to remove the condom, adhesive layer 32 is preferably also capable of releasing from said penis without damaging the penis' skin. These multiple functions are preferably accomplished by using a somewhat weaker adhesive than that used in layer 30. One such suitable adhesive is Medical Transfer Adhesive #1524 made by the 3M Company. The pull strength of Medical Transfer Adhesive #1524 is forty ounces-per-inch, with said pull-strength indicating that it is a weaker adhesive than the adhesive of layer 30. Separation of layer 32 from the skin will preferably be accomplished physically, but it is also possible that a chemical solvent, or other manner of release means, may be employed.

When the condom 1 is made, adhesive layer 30 is applied to the condom and adhesive layer 32 is applied to surface 34 of the backing strip. Then, the backing strip, with adhesive layer 32 facing adhesive layer 30, is pressed onto adhesive layer 30 to create the stacked band of adhesive shown. Alternatively, both adhesive layers can be applied to either the condom or the backing strip.

It should be noted that a band of adhesive is herein defined as a ring of adhesive material and can be composed of one adhesive, or a plurality of adhesives, in any number of layers. However, a significant advantage of having two adhesive layers is that by having a weaker outer layer, one can use a backing strip made of the same expandable material as the membrane 2 of the condom. If just a single layer of adhesive were used, removal of a backing strip made of the same material as the condom could cause the adhesive to pull away from the membrane 2 and leave a gap through which semen could pass. However, a single layer of adhesive may suffice if the backing strip is not made of the same material as membrane 2 and/or if the adhesive and/or backing strip has been modified to facilitate removal of the backing strip and if said adhesive can secure the condom and still allow disengagement from the penis without damaging said penis.

Since the invention calls for a backing strip 28 that can be rolled with the condom, and then expand with said condom during the donning process, said backing strip is preferably flexible and made of a resilient material that enables the backing strip to be expandable. This is preferably achieved by making the backing strip from the same flexible and expandable material as the condom, such as latex. Alternatively, the backing strip can be fashioned from a plurality of interconnected elements that in combination are flexible and expandable, yet can be removed as a single strip. As another alternative, while the backing strip eases a user's ability to unroll the condom from its rolled state, a backing strip may not employed and the multi-layer band of adhesive would be covered by the condom itself until the condom is donned.

The backing strip 28 is preferably of a length whereby it is approximately one-third of an inch longer than the length of the band of adhesive. As a result, after the backing strip is applied to the band of adhesive, a short length of the backing strip will not contact the adhesive and thereby form a pull-tab 36. The pull-tab can then be employed by a user to facilitate removal of the backing strip from the band of adhesive. The pull-tab can also include indicia or a unique coloration that facilitates a user's ability to locate said pull-tab.

Adjacent the band of adhesive is the condom's weakened area 8. The weakened area is designed to enable a user to easily separate the condom's body portion 4 from its cap portion 6. In the embodiment shown in FIGS. 1-7, the weakened area 8 is achieved through the use of a plurality of perforations 38 that extend partially, or completely, through the membrane 2 and thereby locally weaken said membrane. As shown, the perforations are arrayed in a line 40 that extends circumferentially about the condom and effectively defines the border between the cap portion 6 and the body portion 4.

The procedure for donning the condom 1 is extremely simple. Initially, when the condom is first removed from its storage/sale packaging (not shown), the condom would most likely be substantially flat with its body portion and part of its cap portion rolled up.

The user would then prepare the condom for installation by holding the rolled portion of the condom while pulling outwardly on the tip portion 22 of the cap portion until said tip portion moves slightly away from the condom's body portion. This would result in the configuration shown in FIGS. 1 and 2. The user would then continue holding the condom by its rolled portion and begin placing the condom onto the penis.

FIG. 3 shows the condom partially in place on a human penis 42 (shown in phantom). At this point, the condom's cap portion fully covers the glans penis 44, while the condom's body portion is still in a completely rolled state. The cap portion has been unrolled to the point where the band of adhesive is located on an exposed surface and faces outwardly. One should note that at this stage, the band of adhesive is still covered by the backing strip 28. The backing strip's pull-tab 36 is preferably readily accessible and can be easily grasped by a user. The user would next grasp the pull-tab and pull it outwardly until the backing strip is fully removed from the band of adhesive to thereby expose adhesive layer 32.

FIG. 4 shows the next stage in the donning process. As shown, the condom has been further unrolled whereby the adhesive layer 32 now contacts the skin of the penis' shaft 46. At this point, a user would press on the condom in the area that overlies the band of adhesive, thereby forcing the adhesive layer 32 into firm contact with the penis.

It should be noted that the condom 1 is sized whereby when it is made of a resilient material, such as latex, a user will normally stretch the condom during the donning process. This would involve up to about a seventy-five percent increase in the inner diameter of the body portion to easily get it over the glans penis. This is possible in condom 1 since the backing strip 28 is expandable. Once the condom is located on the penis, the condom's membrane will relax to a significant extent, but not a full extent, whereby the inner diameter of the condom's body portion will still be increased by about ten percent over its pre-donning diameter. In this manner, the membrane 2 proximate the weakened area 8 will exert a force on the membrane 2 overlying the band of adhesive that also functions to press the adhesive layer 32 onto the penis.

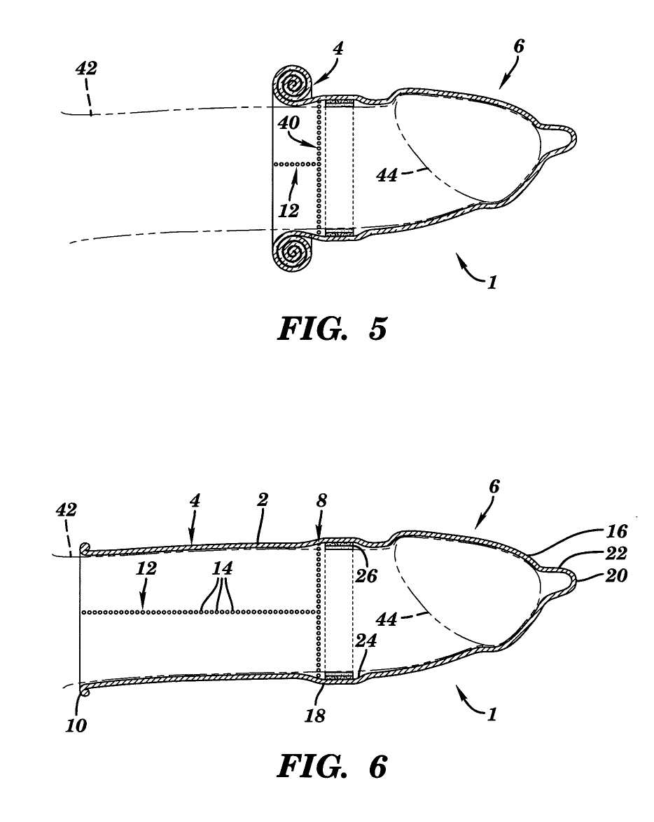

FIG. 5 shows the condom further unrolled from the position it had in FIG. 4. At this point, a user can either remove the condom's body portion, or he can continue to unroll the condom onto the penis.

If the body portion 4 is to be left on the penis whereby the condom 1 will resemble a conventional condom, the user continues to unroll the condom's body portion onto the shaft of the penis until it is substantially fully unrolled. FIG. 6 shows the condom in this substantially fully unrolled state. By leaving the condom's body portion attached to the cap portion, the body portion can aid in maintaining the cap portion in place on the penis. The area of the body portion proximate the weakened area applies pressure on the adjacent part of the cap portion to continually force it into full contact with the penis, thereby keeping the securement pressure at a maximum on the band of adhesive. In this state, the condom provides maximum security for use during sexual intercourse.

A significant advantage of the condom 1 over the prior art is its capacity to be used as a mini-condom that only covers the glans penis. To accomplish this minimalist configuration without having a relatively large rolled-up portion of the condom on the penis, the condom 1 is adapted to facilitate the separation of the condom's body portion from its cap portion.

To remove the body portion 4 when the condom 1 is in the condition shown in FIG. 5, the user holds the condom's cap portion in place and pulls on the condom's rolled body portion, thereby causing the condom to rip circumferentially along the line 40 of perforations 38. The body portion 4 will then separate from the cap portion without disturbing the securement of said cap portion on the penis. Once detached, the body portion 4 can either be stretched over the cap portion and removed from the penis, or the user can apply sideways pressure on the body portion in the area of its line 12 of perforations 14 until said body portion rips along said line 12. An advantage of ripping apart the body portion is that its removal can then be accomplished without any chance of contacting, and possibly dislodging, the condom's cap portion. It should be noted that while line 12 of perforations 14 facilitates removal of the body portion from the penis, said perforations are optional.

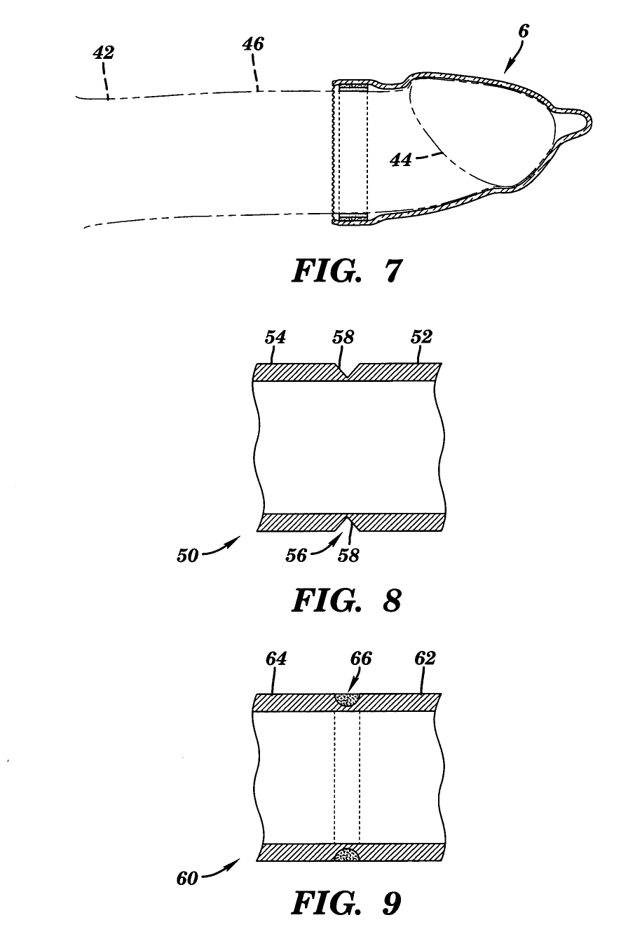

FIG. 7 shows the condom of FIG. 5 after a user has separated the condom's body portion from the cap portion, and then removed said body portion from the penis. At this point, the cap portion is being held onto the penis primarily by the band of adhesive. It should be noted that when the membrane 2 is made from a resilient material, the resiliency of the membrane 2 trying to contract onto the penis will also tend to hold the cap portion to the penis. In this mini-condom version of the invention, most of the penis is left uncovered, thereby maximizing the ability of the penis to receive tactile sensation.

When used as a mini-condom, the cap portion 2 covering the glans penis will function to both receive the semen from the penis and also to prevent transmittal of disease that might occur as a result of fluid transfer between the persons involved in the sexual activity. The condom configuration shown in FIG. 7 is ideal for use during fellatio when the forces on the condom are less than would be applied during intercourse.

FIG. 8 shows a first alternate embodiment of a weakened portion of a condom 50 in accordance with the invention. Condom 50 includes a cap portion 52 (partially shown) having a band 26 of adhesive (not shown), a body portion 54 (partially shown), and wherein said portions are substantially identical to the respective cap and body portions of condom 1. Similar to area 8 of condom 1, there is a weakened area 56 located between the cap and body portions of condom 50. However, in this embodiment, instead of weakening the condom membrane using a series of perforations, area 56 has been weakened by a circumferentially-extending groove 58 in the membrane 2. The groove was preferably produced during the early stages of the condom's fabrication and may have been caused by a knife, laser or other cutting mechanism. As a result of the groove, there is a localized reduction in the thickness of membrane 2 that acts to locally reduce the strength of the membrane and thereby facilitates a user's ability to rip apart the condom at area 56. It should be noted that the groove may only be visible when the condom is in an expanded state.

FIG. 9 shows a second alternate embodiment of a weakened portion of a condom 60 in accordance with the invention. Condom 60 includes a cap portion 62 (partially shown) having a band 26 of adhesive (not shown) and a body portion 64 (partially shown)>Said portions are both substantially identical to the respective portions of condom 1. However, instead of using perforations to weaken the area between the condom's cap and body portions, condom 60 includes a weakened area 66 between said portions that was created by a process that has produced a localized change in the material structure of the membrane 2. The change in the condom material is preferably achieved through the use of a localized heat treatment that makes the condom material in area 66 more brittle than the condom material adjacent to said area. Alternatively, a chemical treatment, such as a weak solvent, can be applied to area 66 to thereby cause a weakening of the molecular bonds in area 66. A user applying a tearing force on condom 60 proximate area 66 will cause the material in area 66 to tear.

It is noted that each of the previously described embodiments of the invention has employed a weakened area between the condom's cap and body portions that extends in a circle oriented perpendicular to a longitudinal axis of the condom. Alternatively, said weakened area can be angled relative to said longitudinal axis.

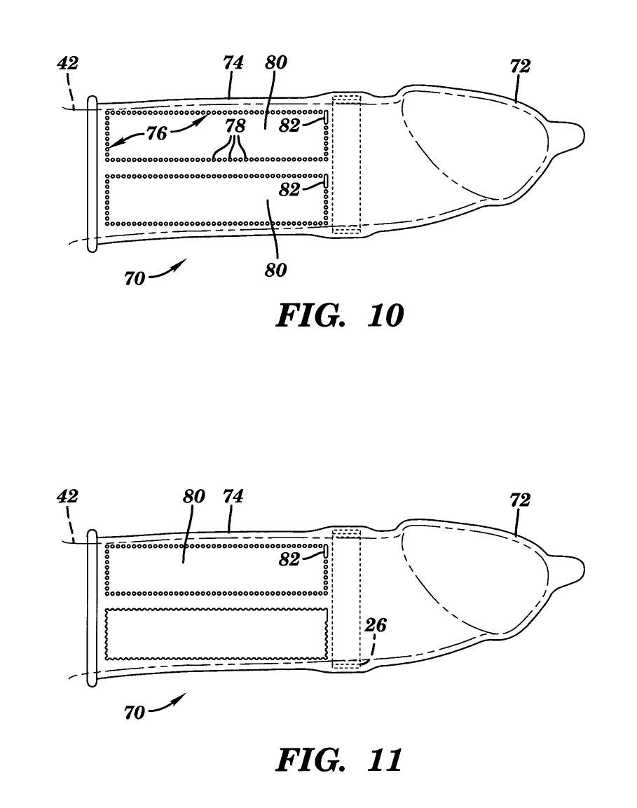

FIG. 10 shows an alternate embodiment of a condom 70 in accordance with the invention. The condom includes a cap portion 72 and a tubular body portion 74. Located in said body portion are lines 76 of perforations 78 that are preferably arrayed to form a plurality of rectangular window portions 80. Said lines of perforations locally weaken the condom material and thereby enable easy removal of any of the window portions 80. It should be noted that the condom may include one, or a plurality of such window portions in its body portion. In addition, each line of perforations can include a slit portion 82 that can act as a starting point to facilitate disengaging a window portion 80 from the condom's body portion. As in the other embodiments of the invention taught herein, the cap portion includes a band 26 of adhesive.

FIG. 11 shows the condom of FIG. 10 after one of the window portions 80 has been removed. In this manner, the condom provides increased exposure of the penis 42 compared to prior art conventional condoms, while the band 26 of adhesive and the resiliency of the condom material of the condom's cap and body portions maintains the condom's cap portion in position on the penis.

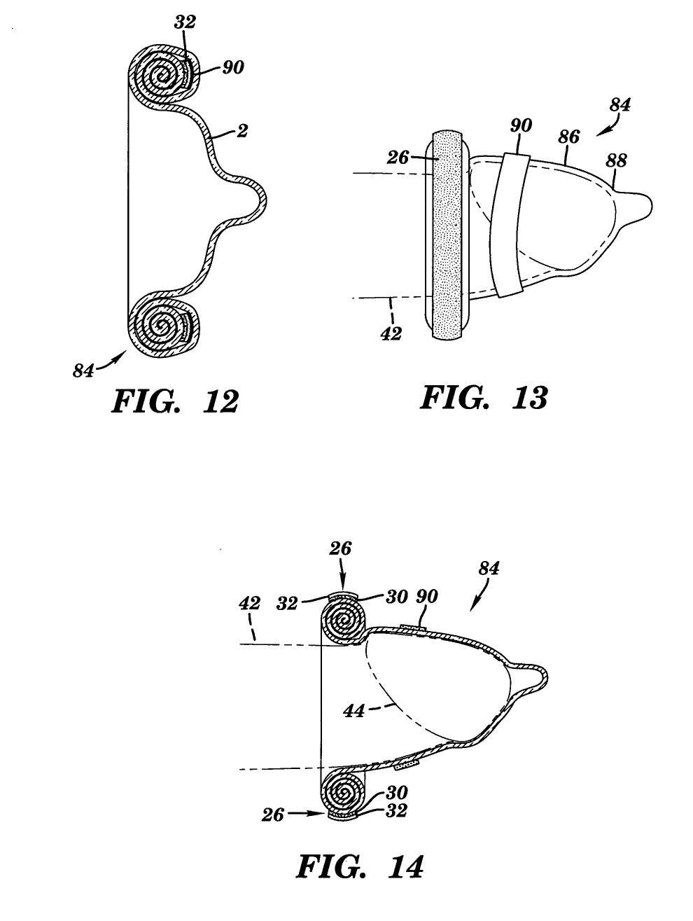

FIG. 12 provides a cross-sectional view of another condom 84 in accordance with the invention. Condom 84 is preferably substantially identical to condom 1, except that it has been modified in a manner whereby a backing strip is not required to cover the band 26 of adhesive. In this embodiment, a portion of the surface 86 of the condom's cap portion 88 includes a circular band 90 of a non-stick material, such as silicone or PTFE, bonded to the condom membrane 2. The width of said band of non-stick material is preferably equal to, or slightly greater than, the width of the band 26 of adhesive. The location of band 90 is such that when the condom is in the rolled state shown in FIG. 12, the outer adhesive layer 32 of band 26 releasably contacts band 90. As a result, when the condom is unrolled from the state shown in FIG. 12 to the further unrolled state shown in side view in FIG. 13 and cross-sectional view in FIG. 14, band 26 of adhesive does not stick to band 90. From the state shown in FIGS. 13 and 14, the condom can be further unrolled whereby band 26 of adhesive is placed into contact with a user's penis 42.

The preferred embodiments of the invention disclosed herein have been discussed for the purpose of familiarizing the reader with the novel aspects of the invention. Although preferred embodiments of the invention have been shown and described, many changes, modifications and substitutions may be made by one having ordinary skill in the art without necessarily departing from the spirit and scope of the invention as described in the following claims.

I claim:

1. A condom comprising:
a tubular body portion made of a flexible material, wherein said body portion includes first and second end portions, and wherein said first end portion defines an opening through which a user can insert a penis;
a cap portion made of a substantially fluid impermeable material, wherein said cap portion includes a closed, substantially dome-shaped first end portion and a second end portion that is secured to the second end portion of said body portion at a weakened area of said condom, wherein a band of adhesive is located on a surface of said cap portion proximate the cap portion's second end portion whereby when said condom is in place on a user's penis, said band of adhesive can directly contact said penis in a manner whereby it creates a fluid barrier that extends completely around said penis; and
wherein said weakened area of said condom is weaker than adjacent areas of said body portion and said cap portion to thereby facilitate separation of the body portion from said cap portion.

2. The condom of claim 1 wherein said cap portion includes a tubular fluid reservoir portion.

3. The condom of claim 1 wherein said weakened area is achieved through the use of a plurality of perforations that extend at least partially through a membrane that forms said condom in said weakened area.

4. The condom of claim 1 wherein a weakened area also extends along a side of the body portion in a direction parallel to a longitudinal axis of said condom.

5. The condom of claim 1 wherein said condom is fashioned from a membrane that is substantially uniform in thickness except in said weakened area where said thickness is reduced.

6. The condom of claim 1 wherein said condom is made of a flexible membrane and wherein said weakened area is formed by creating a groove in said membrane.

7. The condom of claim 1 wherein said band of adhesive comprises first and second layers, wherein said first layer is made of a first adhesive that is secured to a surface of said condom, and wherein said second layer is made of a second adhesive that is weaker than said first adhesive.

8. The condom of claim 7 wherein said second adhesive is adapted for releasable contact with a human penis.

9. The condom of claim 1 wherein said band of adhesive is covered by a removable backing strip.

10. The condom of claim 9 wherein said backing strip is expandable.

11. The condom of claim 10 wherein said condom and said backing strip are both made of the same material.

12. The condom of claim 1 wherein said cap portion has a length whereby when a typical human adult's penis having a glans penis and a shaft is inserted through the opening in the body portion's first end portion, said condom can be positioned to substantially fully receive said penis in a manner whereby the glans penis is at least partially received within the condom's cap portion.

13. The condom of claim 1 wherein once a typical human adult's penis is inserted through the opening in the body portion's first end portion, a user can detach said body portion from said cap portion at said weakened area and then remove said body portion from said penis while leaving said cap portion on said penis.

14. The condom of claim 1 wherein said condom, when in a fully unrolled state, has a length, and wherein said body portion is responsible for a major portion of said length and said cap portion is responsible for a minor portion of said length.

15. A condom comprising:
a tubular body portion, wherein said body portion includes an end portion that defines an opening through which a user can insert a penis, and wherein said body portion is made of a flexible material; and
a cap portion that has a closed, substantially dome-shaped first end portion and a second end portion secured to the body portion and that is a continuation of said body portion, wherein said cap portion features a band of adhesive covered by an expandable backing strip, wherein said band of adhesive is located on a surface of said cap portion whereby when said condom is in place on a user's penis and said backing strip is removed, said band of adhesive can be placed into direct contact with said penis in a manner that creates a fluid barrier that extends completely around said penis, and wherein said cap portion is made of a material that is both flexible and fluid impermeable.

16. The condom of claim 15 wherein said condom and said backing strip are both made of the same material.

17. The condom of claim 15 wherein said backing strip has a length whereby when it is placed onto said band of adhesive, a first portion of said backing strip overlaps a second portion of said backing strip in a manner whereby said first portion of said backing strip can act as a pull-tab to facilitate removal of said backing strip from said band of adhesive.

18. A condom comprising:
a tubular body portion that includes an end that defines an opening through which a user can insert a penis;
a cap portion made of a substantially fluid impermeable material, wherein said cap portion has first and second end portions, wherein said first end portion is substantially dome-shaped and forms a closed end of said condom, wherein said second end portion is secured to said body portion, wherein said cap portion features a band of adhesive located whereby said band of adhesive can completely encircle a portion of a human penis when said condom is in place in a predetermined manner on said penis and thereby create a fluid barrier; and
wherein said band of adhesive comprises a first layer of adhesive secured to a second layer of adhesive, wherein said first layer of adhesive employs a first adhesive that has a strength of adhesion, and wherein said second layer of adhesive employs a second adhesive that has a strength of adhesion greater than that of the first adhesive, wherein said second layer of adhesive directly contacts a surface of said cap portion.

19. The condom of claim 18 wherein said first layer of adhesive is adapted to removably secure an expandable backing strip, and wherein when said backing strip is pulled away from said band of adhesive, said second layer of adhesive will remain secured to said condom.

20. The condom of claim 18 wherein said first layer of adhesive is adapted to removably secure a backing strip, wherein when said backing strip is pulled away from said band of adhesive, said first layer of adhesive will remain secured to said second layer of adhesive, wherein when said condom is in place on a human penis, said first layer of adhesive will directly contact said penis and wherein said first layer of adhesive is sufficiently weak whereby once it has been placed into contact with a user's penis, a user can later disengage said first layer of adhesive from said penis without damaging said penis.

21. The condom of claim 18 wherein said body portion includes a removable window portion defined by a shaped weakened area of said body portion and wherein when said condom is in place on a human penis, a user can remove said window portion from said body portion and thereby expose a portion of said penis located between said end of said body portion and said band of adhesive.

22. The condom of claim 18 wherein when said condom is in place on a penis, said condom has an inwardly-facing surface and an outwardly-facing surface, wherein said band of adhesive is located on said inwardly-facing surface, wherein a material to which said band of adhesive will not adhere is located on said outwardly-facing surface and wherein when said condom is in a rolled-up state, said band of adhesive contacts said material.
